(19) United States
(12) Patent Application Publication
(10) Pub. No.: US 2026/0174941 A1
(43) Pub. Date: Jun. 25, 2026

(54) METHODS OF REDUCING FILL TIME BY PREHEATING THE SOLUTION USING AN INLINE HEATER

(71) Applicant: GAMBRO LUNDIA AB, Lund (SE)

(72) Inventors: Shashanka Jain, Bangalore (IN); Hemanth Ravikumar, Bangalore (IN)

(21) Appl. No.: 19/429,716

(22) Filed: Dec. 22, 2025

(30) Foreign Application Priority Data
Dec. 24, 2024 (IN) 202441102678

(51) Int. Cl. A61M 1/14; A61M 1/28

(52) U.S. Cl. CPC A61M 1/159; A61M 1/153; A61M 1/154; A61M 1/288; A61M 1/287; A61M 2205/3334; A61M 2205/3344; A61M 2205/3372; A61M 2205/3379

(57) ABSTRACT

The present disclosure sets forth a peritoneal dialysis (“PD”) system, which allows the lowest operating ambient temperature of the PD fluid to be decreased without modifying the heater power or the flow rate during treatment. The system includes a PD machine or cycler. Fresh PD fluid is delivered via a single or dual lumen patient line to the patient and is first heated to a body fluid temperature, e.g., 37° C. Before fill, the PD fluid is preheated by pumping the fresh PD fluid from a solution bag, through the inline heater, into an empty solution bag, and back until the fresh PD fluid is heated to a pre-determined temperature. This allows the initial temperature of the fresh PD fluid in the solution bag to be lower.

### CROSS-REFERENCE TO RELATED APPLICATIONS

The present application claims the benefit of Indian Provisional Application No. 20/244,1102678, entitled METHODS OF REDUCING FILL TIME BY PREHEATING THE SOLUTION USING AN INLINE HEATER and filed Dec. 24, 2024, the contents of which are hereby incorporated by reference in their entirety.

### TECHNICAL FIELD

The present disclosure relates generally to medical fluid treatments, and in particular to dialysis fluid treatments that require a certain medical fluid temperature for treatment.

### BACKGROUND

Due to various causes, a person's renal system can fail. Renal failure produces several physiological derangements. For instance, it is no longer possible to balance water and minerals or to excrete daily metabolic load. Additionally, toxic end products of metabolism, such as urea, creatinine, uric acid, and others, may accumulate in a patient's blood and tissue.

Reduced kidney function and, above all, kidney failure is treated with dialysis. Dialysis removes waste, toxins, and excess water from the body that normal functioning kidneys would otherwise remove. Dialysis treatment for the replacement of kidney functions is critical to many people because the treatment is lifesaving.

One type of kidney failure therapy is Hemodialysis (“HD”), which in general uses diffusion to remove waste products from a patient's blood. A diffusive gradient occurs across a semi-permeable dialyzer between the blood and an electrolyte solution, called dialysate or dialysis fluid, to cause diffusion.

Hemofiltration (“HF”) is an alternative renal replacement therapy that relies on a convective transport of toxins from a patient's blood. HF is accomplished by adding substitution or replacement fluid to an extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD, HF, and HDF treatments occur in centers. A trend towards home hemodialysis (“HHD”) exists today in part because HHD can be performed daily, offering therapeutic benefits over in-center hemodialysis treatments, which occur typically bi- or tri-weekly. Studies have shown that more frequent treatments remove more toxins and waste products and render less interdialytic fluid overload than a patient receiving less frequent but perhaps longer treatments. A patient receiving more frequent treatments does not experience as much of a down cycle (swings in fluids and toxins) as does an in-center patient, who has built-up two or three days' worth of toxins prior to a treatment. In certain areas, the closest dialysis center can be many miles from the patient's home, causing door-to-door treatment time to consume a large portion of the day. Treatments in centers close to the patient's home may also consume a large portion of the patient's day. HHD can take place overnight or during the day while the patient relaxes, works or is otherwise productive.

Another type of kidney failure therapy is peritoneal dialysis (“PD”), which infuses a dialysis solution, also called dialysis fluid, into a patient's peritoneal chamber via a catheter. The dialysis fluid is in contact with the peritoneal membrane in the patient's peritoneal chamber. Waste, toxins, and excess water pass from the patient's bloodstream, through the capillaries in the peritoneal membrane, and into the dialysis fluid due to diffusion and osmosis, i.e., an osmotic gradient occurs across the membrane. An osmotic agent in the PD dialysis fluid provides the osmotic gradient. Used or spent dialysis fluid is drained from the patient, removing waste, toxins, and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis, and continuous flow peritoneal dialysis (“CFPD”). CAPD is a manual dialysis treatment. Here, the patient manually connects an implanted catheter to a drain to allow used or spent dialysis fluid to drain from the peritoneal chamber. The patient then switches fluid communication so that the patient catheter communicates with a bag of fresh dialysis fluid to infuse the fresh dialysis fluid through the catheter and into the patient. The patient disconnects the catheter from the fresh dialysis fluid bag and allows the dialysis fluid to dwell within the peritoneal chamber, where the transfer of waste, toxins, and excess water takes place. After a dwell period, the patient repeats the manual dialysis procedure, for example, four times per day. Manual peritoneal dialysis requires a significant amount of time and effort from the patient, leaving ample room for improvement.

Automated peritoneal dialysis (“APD”) is similar to CAPD in that the dialysis treatment includes drain, fill, and dwell cycles. Automated PD machines, however, perform the cycles automatically, typically while the patient sleeps. The PD machines free patients from having to manually perform the treatment cycles and from having to transport supplies during the day. The PD machines connect fluidly to an implanted catheter, to a source or bag of fresh dialysis fluid and to a fluid drain. The PD machines pump fresh dialysis fluid from a dialysis fluid source, through the catheter and into the patient's peritoneal chamber. The PD machines also allow for the dialysis fluid to dwell within the chamber and for the transfer of waste, toxins, and excess water to take place. The source may include multiple liters of dialysis fluid including several solution bags.

Each of the above-identified dialysis modalities, except for CAPD (which typically does not involve machinery), heats the dialysis fluid prior to use for treating the patient. The dialysis fluid is typically heated to body temperature or approximately 37° C. so that the patient does not experience a thermal shock when the dialysis fluid comingles with the patient's blood or is delivered to the patient's peritoneal cavity.

Typically, in PD systems, fluid is pumped from a solution bag, through an inline heater where the fluid is heated to body temperature, and into the patient. The PD fluid may need to be a certain ambient temperature in order for the inline heater to heat the fluid to body temperature at a desired flow rate. However, in some countries, patients do not turn on the heater at night while sleeping (i.e. when most patients undergo therapy), causing the ambient temperature of the PD fluid to drop. Currently, if the lowest operating ambient temperature is below a certain threshold, either the flow rate has to be reduced (i.e. therapy time increases) to achieve the 37° C. fill temperature or the heater power has to be increased. Increasing heater power may result in an increased cost and size.

It is desirable for the PD machine to heat the dialysis fluid to body temperature while still maintaining a desired flow rate and power requirements of the heater. As such, devices and methods for preheating the PD solution using an inline heater are accordingly needed.

### SUMMARY

The present disclosure sets forth an automated peritoneal dialysis (“PD”) system, which allows the lowest operating ambient temperature of the PD fluid to be decreased without modifying the heater power or the flow rate during treatment. The system includes a PD machine or cycler. The PD machine is configured to deliver fresh, heated PD fluid to a patient at, for example, 14 kPa (2.0 psig) or higher. The PD machine is capable of removing used PD fluid or effluent from the patient at, for example, −9 kPa (−1.3 psig) or higher. Fresh PD fluid is delivered via a single or dual lumen patient line to the patient and is first heated to a body fluid temperature, e.g., 37° C. Before fill, the PD fluid may be pre-heated by pumping the fresh PD fluid from a solution bag, through the inline heater, into an empty solution bag, and back until the fresh PD fluid is heated to a pre-determined temperature. This allows the initial temperature of the fresh PD fluid in the solution bag to be lower.

In light of the disclosure set forth herein, and without limiting the disclosure in any way, in a first aspect of the present disclosure, which may be combined with any other aspect, or portion thereof, provides a peritoneal dialysis (“PD”) system including a housing; a PD fluid pump housed by the housing; a source of fresh PD fluid; an inline heater positioned and arranged to heat the fresh PD fluid; a fluid line in fluid communication with the source of fresh PD solution, the inline heater, and the PD fluid pump; a temperature sensor configured to sense a temperature of the source of the fresh PD fluid; and a control unit configured to control the PD fluid pump. The control unit is further configured to receive a sensed temperature of the fresh PD fluid from the temperature sensor during a dwell phase or a priming phase of peritoneal dialysis treatment, determine whether the sensed temperature is below a pre-determined threshold, and cause the PD fluid pump to pump the fresh PD fluid through the fluid line and through the inline heater when the sensed temperature is below the pre-determined threshold.

According to a second aspect of the present disclosure, the source of fresh PD fluid includes a first solution container, and the PD system further includes a second solution container. The control unit is configured to cause the PD fluid pump to pump the fresh PD fluid from the first solution container, through the fluid line, through the inline heater, and to the second solution container when the sensed temperature is below the pre-determined threshold.

According to a third aspect of the present disclosure, the control unit is further configured to cause the PD fluid pump to reverse the flow of the PD fluid pump and pump the fresh PD fluid from the second solution container to the first solution container and back until the sensed temperature is above the pre-determined threshold.

According to a fourth aspect of the present disclosure, the temperature sensor is located at the source of the fresh PD fluid.

According to a fifth aspect of the present disclosure, the temperature sensor is located along the fluid line prior to the inline heater.

According to a sixth aspect of the present disclosure, the control unit is further configured to cause the PD fluid pump to begin a patient fill when the sensed temperature is above the pre-determined threshold.

According to a seventh aspect of the present disclosure, the control unit is further configured to cause the PD fluid pump to begin the patient fill at a flow rate above 300 ml/min.

According to an eighth aspect of the present disclosure, the inline heater has a power of less than 650 W.

According to a ninth aspect of the present disclosure, the pre-determined threshold is between 15° C. and 30° C.

A tenth aspect of the present disclosure provides a peritoneal dialysis (“PD”) method for preheating a PD fluid. The method includes sensing a temperature, via a temperature sensor controlled by a control unit of a PD machine, of fresh PD fluid in a container; determining, via the control unit, whether the sensed temperature is below a pre-determined threshold; causing a PD fluid pump of the PD machine, via the control unit, to pump the fresh PD fluid from the container through an inline heater and back when the sensed temperature is below the pre-determined threshold; and continuing to cause the PD fluid pump, via the control unit, to pump the fresh PD fluid from the container through the inline heater and back until the sensed temperature is above the pre-determined threshold.

According to an eleventh aspect of the present disclosure, the method further includes causing a PD pump, via the control unit, to begin a patient fill when the sensed temperature is above the pre-determined threshold.

According to a twelfth aspect of the present disclosure, the patient fill occurs at a flow rate of above 300 ml/min.

According to a thirteenth aspect of the present disclosure, the sensing the temperature occurs during a priming phase of a PD treatment as determined by the control unit.

According to a fourteenth aspect of the present disclosure, the sensing the temperature occurs during a dwell phase of a PD treatment as determined by the control unit.

According to a fifteenth aspect of the present disclosure, the method further includes closing a patient line valve, via the control unit, prior to causing the PD fluid pump to pump the fresh PD fluid from the container.

According to a sixteenth aspect of the present disclosure, the sensing the temperature occurs between 10 minutes and 20 minutes before an end of the dwell phase.

According to a seventeenth aspect of the present disclosure, the pre-determined threshold is between 15° C. and 30° C.

According to an eighteenth aspect of the present disclosure, the inline heater has a power of less than 650 W.

A nineteenth aspect of the present disclosure provides a peritoneal dialysis (“PD”) system including a housing; a PD fluid pump housed by the housing; a first container comprising a fresh PD fluid; a second container; an inline heater positioned and arranged to heat the fresh PD fluid; a fluid line in fluid communication with the first container, the inline heater, the PD fluid pump, and the second container; a temperature sensor configured to sense a temperature of the fresh PD fluid; and a control unit configured to control the PD fluid pump. The control unit is further configured to receive a sensed temperature of the fresh PD fluid from the temperature sensor during a dwell phase or a priming phase of peritoneal dialysis treatment, determine whether the sensed temperature is below a pre-determined threshold, and cause the pump to pump the fresh PD fluid from the first container, through the fluid line, and through the inline heater when the sensed temperature is below the pre-determined threshold.

According to a twentieth aspect of the present disclosure, the control unit is further configured to cause the PD fluid pump to reverse the flow of the PD fluid pump and pump the fresh PD fluid from the second container to the first container and back until the sensed temperature is above the pre-determined threshold.

In a twenty-first aspect of the present disclosure, which may be combined with any other aspect, or portion thereof, any of the features, functionality and alternatives described in connection with any one or more of FIGS. 1 to 3 may be combined with any of the features, functionality and alternatives described in connection with any other of FIGS. 1 to 3.

In light of the above aspects and present disclosure set forth herein, it is an advantage of the present disclosure to provide a system and method for preheating PD fluid so that a lowest operating ambient temperature of the PD fluid may be lower.

It is another advantage of the present disclosure to provide a PD fluid system that allows a patient to sleep without heat while still maintaining a desired therapy time.

It is another advantage of the present disclosure to provide a PD fluid system that allows a patient to sleep without heat and without increasing the power requirements of the inline heater.

It is yet another advantage of the present disclosure to provide a PD fluid system where the flow rate during fill is not limited to heater power, which can result in reduced therapy time.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a fluid flow schematic of one embodiment for a medical fluid, e.g., PD fluid, system that is set for treatment.

FIGS. 2A and 2B are fluid flow schematics of one embodiment for a medical fluid e.g., PD fluid system configured for preheating the medical fluid prior to treatment or between phases of treatment. FIG. 2A illustrates the forward direction and FIG. 2B illustrates the reverse flow.

FIG. 3 is a process flow diagram illustrating one embodiment for preheating PD fluid.

### DETAILED DESCRIPTION

### System Overview

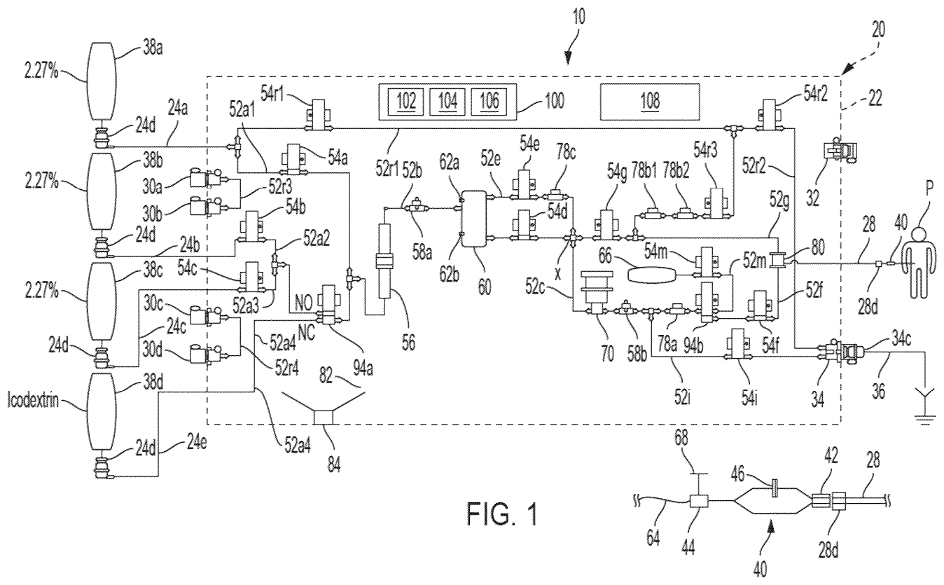

Referring now to the drawings and in particular to FIG. 1, a peritoneal dialysis (“PD”) system 10 according to the present disclosure is illustrated. System 10 includes a PD machine or cycler 20 and a control unit 100 having one or more processor 102, one or more memory 104, video controller 106, and user interface 108. The example user interface 108 may alternatively or additionally be a remote user interface, e.g., via a tablet or smartphone. The control unit 100 may also include a transceiver and a wired or wireless connection to a network (not illustrated), e.g., the internet, for sending treatment data to and receiving prescription instructions/changes from a doctor's or clinician's server interfacing with a doctor's or clinician's computer. The control unit 100 in an embodiment controls all electrical, fluid flow, and heating components of the system 10 and receives outputs from sensors of the system 10. System 10 in the illustrated embodiment includes durable and reusable components that contact fresh and used PD fluid, which necessitates that the PD machine or cycler 20 be disinfected between treatments, e.g., via heat disinfection.

System 10 in FIG. 1 includes an inline resistive heater 56, reusable supply lines or tubes 52a1 to 52a4 and 52b, air trap 60 operating with respective upper and lower level sensors 62a and 62b, air trap valve 54d, vent valve 54e located along vent line 52e, reusable line or tubing 52c, PD fluid pump 70, temperature sensors 58a and 58b, pressure sensors 78a, 78b1, 78b2 and 78c, reusable patient tubing or lines 52f and 52g having respective valves 54f and 54g, dual lumen reusable patient line 28, a hose reel 80 for retracting patient line 28, reusable drain tubing or line 52i extending to drain line connector 34 and having a drain line valve 54i, and reusable recirculation disinfection tubing or lines 52r1 and 52r2 operating with respective disinfection valves 54r1 and 54r2. A third recirculation or disinfection tubing or line 52r3 extends between disinfection connectors 30a and 30b for use during disinfection. A fourth recirculation or disinfection tubing or line 52r4 extends between disinfection connectors 30c and 30d for use during disinfection.

System 10 further includes PD fluid containers or bags 38a to 38c (e.g., holding the same or different formulations of PD fluid), which connect to distal ends 24d of reusable PD fluid lines 24a to 24c, respectively. System 10 further includes a fourth PD fluid container or bag 38d that connects to a distal end 24d of reusable PD fluid line 24e. Fourth PD fluid container or bag 38d may hold the same or different type (e.g., icodextrin) of PD fluid than provided in PD fluid containers or bags 38a to 38c. Reusable PD fluid lines 24a to 24c and 24e extend in one embodiment through apertures defined or provided by the housing 22 of the cycler 20.

System 10 in the illustrated embodiment includes four disinfection connectors 30a to 30d for connecting to distal ends 24d of reusable PD fluid lines 24a to 24c and 24e, respectively, during disinfection. System 10 also provides a patient line connector 32 that includes an internal lumen, e.g., a U-shaped lumen, which for disinfection directs fresh or used dialysis fluid from one PD fluid lumen of a connected distal end 28d of dual lumen reusable patient line 28 into the other PD fluid lumen. Reusable supply tubing or lines 52al to 52a4 communicate with reusable supply lines 24a to 24c and 24e, respectively. Reusable supply tubing or lines 52al to 52a3 operate with valves 54a to 54c, respectively, to allow PD fluid from a desired PD fluid container or bag 38a to 38c to be pulled into cycler 20. Three-way valve 94a in the illustrated example allows for control unit 100 to select between (i) 2.27% (or other) glucose dialysis fluid from container or bag 38b or 38c and (ii) icodextrin from container or bag 38d. In the illustrated embodiment, icodextrin from container or bag 38d is connected to the normally closed port of three-way valve 94a.

System 10 is constructed in one embodiment such that drain line 52i during a patient fill is fluidly connected downstream from PD fluid pump 70. In this manner, if drain valve 54i fails or somehow leaks during the patient fill of patient P, fresh PD fluid is pushed down disposable drain line 36 instead of used PD fluid potentially being pulled into pump 70. Disposable drain line 36 is in one embodiment removed for disinfection, wherein drain line connector 34 is capped via a cap 34c to form a closed disinfection loop. PD fluid pump 70 may be an inherently accurate pump, such as a piston pump, or less accurate pump, such as a gear pump that operates in cooperation with a flowmeter (not illustrated) to control fresh and used PD fluid flowrate and volume. In some embodiments, the PD fluid pump 70 may be a peristaltic pump.

System 10 may further include a leak detection pan 82 located at the bottom of housing 22 of cycler 20 and a corresponding leak detection sensor 84 outputting to control unit 100. In the illustrated example, system 10 is provided with an additional pressure sensor 78c located upstream of PD fluid pump 70, which allows for the measurement of the suction pressure of pump 70 to help control unit 100 more accurately determine pump volume. Additional pressure sensor 78c in the illustrated embodiment is located along vent line 52e, which may be filled with air or a mixture of air and PD fluid, but which should nevertheless be at the same negative pressure as PD fluid located within PD fluid line 52c.

System 10 in the example of FIG. 1 includes redundant pressure sensors 78b1 and 78b2, the output of one of which is used for pump control while the output of the other pressure sensor is a safety or watchdog output to make sure the control pressure sensor is reading accurately. Pressure sensors 78b1 and 78b2 are located along a line including a third recirculation valve 54r3.

System 10 in the example of FIG. 1 further includes a source of acid, such as a citric acid container or bag 66. Citric acid container or bag 66 is in selective fluid communication with second three-way valve 94b via a citric acid valve 54m located along a citric acid line 52m. Citric acid line 52m is connected in one embodiment to the normally closed port of second three-way valve 94b, so as to provide redundant valves between citric acid container or bag 66 and the PD fluid circuit during treatment. The redundant valves ensure that no citric (or other) acid reaches the treatment fluid lines during treatment. Citric (or other) acid is instead used during disinfection.

Control unit 100 in an embodiment uses feedback from any one or more of pressure sensors 78a to 78c to enable PD machine 20 to deliver fresh, heated PD fluid to the patient at, for example, 14 kPa (2.0 psig) or higher. The pressure feedback is used to enable PD machine 20 to remove used PD fluid or effluent from the patient at, for example, −9 kPa (−1.3 psig) or higher. The pressure feedback may be used in a proportional, integral, derivative (“PID”) pressure routine for pumping fresh and used PD fluid at a desired positive or negative pressure.

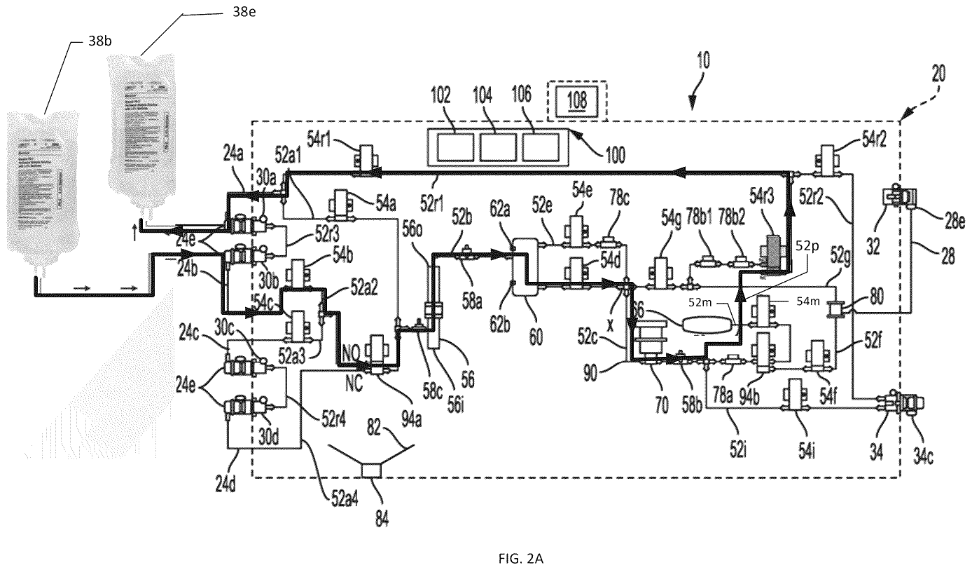

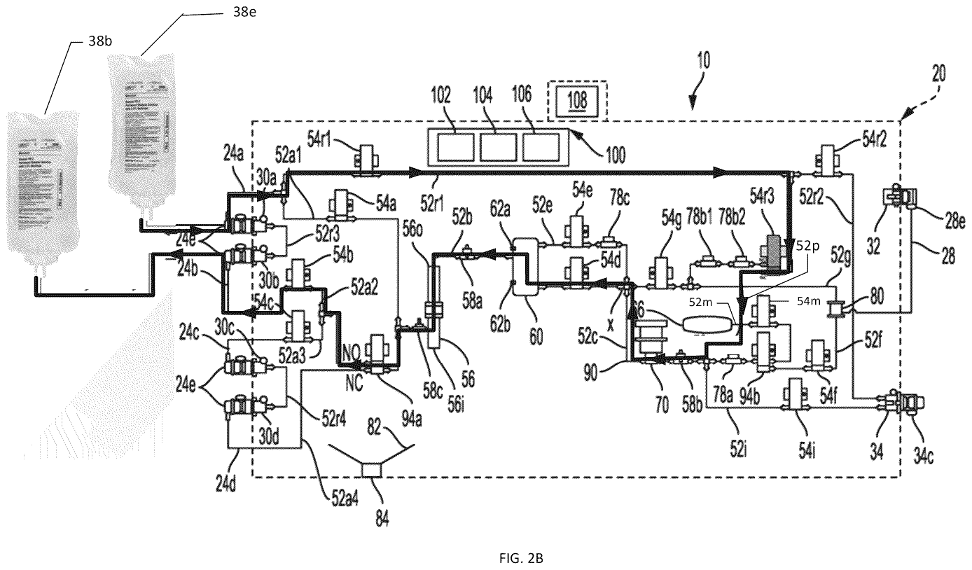

FIG. 1 illustrates system 10 setup for treatment with PD fluid containers or bags 38a to 38d connected via reusable, flexible PD fluid lines 24a to 24d, respectively. Dual lumen patient line 28 is connected to patient P via disposable filter set 40. Disposable drain line 36 is connected to drain line connector 34. In FIGS. 1 and 2, PD machine or cycler 20 of system 10 is configured to perform multiple patient drains, patient fills, patient dwells, and a priming procedure, as part of or in preparation for treatment.

For example, during patient fills, the PD machine or cycler 20 is configured to pump fresh dialysis fluid from PD fluid containers or bags 38a to 38d, through the patient line 28, through the disposable filter set, and into the patient's peritoneal chamber. The PD machines also allow for the dialysis fluid to dwell within the chamber and for the transfer of waste, toxins, and excess water to take place. The source may include multiple liters of dialysis fluid including several solution bags. During patient drain, the PD machine or cycler 20 pumps used or spent dialysate from the patient's peritoneal cavity, though the disposable transfer set, through the patient line, and to the drain.

Inline resistive heater 56 under control of control unit 100 is capable of heating fresh PD fluid to body temperature, e.g., 37° C., for delivery to patient P at a desired flowrate. Control unit 100 in an embodiment uses feedback from temperature sensor 58a in a PID temperature routine for pumping fresh PD fluid to patient P at a desired temperature.

FIG. 1 also illustrates that system 10 includes and uses a disposable filter set 40, which communicates fluidly with the fresh and used PD fluid lumens of dual lumen patient line 28. Disposable filter set 40 includes a disposable connector 42 that connects to a distal end 28d of reusable patient line 28. Disposable filter set 40 also includes a connector 44 that connects to the patient's transfer set. Disposable filter set 40 further includes a sterilizing grade filter membrane 46 that further filters fresh PD fluid. Disposable filter set 40 is provided in one embodiment as a last chance filter for PD machine 20, which has been heat disinfected between treatments. Any pathogens that may remain after disinfection, albeit unlikely, are filtered from the PD fluid via the sterilizing grade filter membrane 46 of disposable filter set 40.

### Fluid Pre-Heating

As described previously, fresh PD fluid is delivered via the patient line 28 and is first heated to a body fluid temperature, e.g., 37° C. using inline heater 56. Before patient fill, such as during priming or the dwell phase, the system 10 senses the PD fluid temperature, such as through temperature sensor 58a, 58b, or an additional temperature sensor. When the temperature of the PD fluid is below a pre-determined threshold, then corrective action needs to be taken to ensure the inline heater 56 is able to adequately heat the PD fluid to body temperature before reaching the patient. Corrective measures may include lowering the pump speed. However, decreasing the pump speed results in longer therapy times, which is disadvantageous for the patient. Further corrective measures may include raising the power of the heater. However, increased heating power requirements may be costly and increase the size of the PD system.

System 10 and associated methods solve the potential low temperature problem by programming a control unit 100 to automatically initiate a preheating operation of the PD fluid before patient fill, such as during priming or dwell. FIGS. 2A and 2B illustrate flow schematics of one embodiment of the PD system 10 with a pathway for preheating fresh PD fluid before patient fill. The control unit 100 in an embodiment uses feedback from a temperature sensor to automatically initiate a push pull operation for preheating the fresh PD fluid before fill.

For example, the control unit 100 receives a temperature of the PD fluid from a temperature sensor. In some embodiments, the temperature sensor is located in the fresh PD solution container or a fluid line extending from the fresh PD solution container (e.g. reusable supply line 24b or reusable supply line 52a2). When the temperature sensor is located along a fluid line (e.g. reusable supply line 24b or reusable supply line 52a2), the control unit 100 may cause the pump 70 to pump an amount of fluid from the fresh PD solution container 38b into the fluid line prior to the temperature sensor sensing and outputting a temperature to the control unit 100. In some embodiments, the control unit 100 causes the pump 70 to pump an amount of fluid from the fresh PD solution container 38b periodically to measure the temperature. This may ensure that the temperature sensed by the temperature sensor is representative of the temperature of the fresh PD solution in the solution container 38b.

When the sensed temperature is below a pre-determined threshold, the control unit 100 will initiate the push pull operation for preheating the fluid until the PD fluid temperature is above the pre-determined threshold. In some embodiments, the pre-determined temperature threshold may be the lowest ambient temperature that the PD fluid may be for the inline heater 56 to heat the PD fluid to body temperature at a desired flow rate and using a desired heater power. For example, the pre-determined temperature threshold may be between 15° C. and 30° C., such as 15° C., 20° C., 25° C., 30° C., or any suitable temperature.

As illustrated in the depicted embodiment, the PD system 10 may include an additional pathway 52p after the pump 70 from reusable line 52c to the recirculation valve 54r3. Additionally, the third recirculation valve 54r3 may include a three-way valve. In some embodiments, a full or partially full solution bag 38e is connected to reusable PD fluid line 24e. The control unit 100 may signal the pump 70 to empty the full or partially full solution bag 38e to drain prior to beginning the push pull operation or prior to the dwell or priming phase. In some embodiments, this occurs before the last fill of the treatment or any other fill cycle of treatment. In some embodiments, an empty solution bag 38e is connected to reusable PD fluid line 24e. The empty solution bag 38e may be from a previous fill cycle.

PD fluid may travel from solution bag 38b through the inline heater 56 to solution bag 38e (FIG. 2A) and back (FIG. 2B) during the push pull operation to preheat the PD fluid. For example, when the temperature of the PD fluid is below the pre-determined threshold, the control unit 100 causes third recirculation valve 54r3 to open. Further, the control unit 100 signals the pump 70 to pump the PD fluid from the PD solution bag 38b through inline heater 56, through third recirculation valve 54r3, through reusable line 52r1, to the additional solution bag 38e (e.g. an empty solution bag) as shown in FIG. 2A. As PD fluid travels through the inline heater 56, the PD fluid is heated.

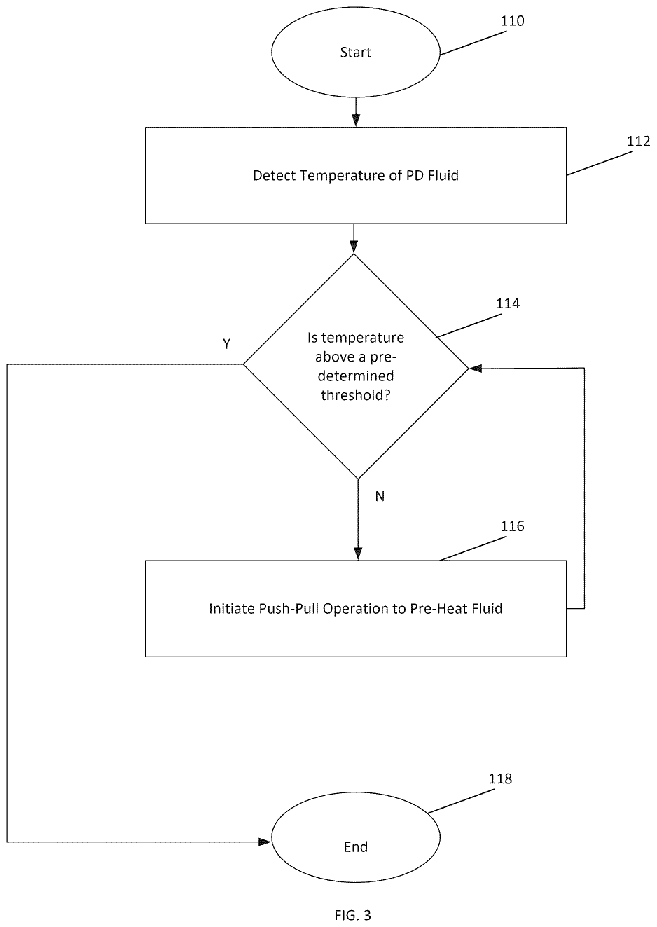

After a desired amount of fluid has been pumped from the solution bag 38b, the control unit 100 causes the pump 70 to reverse direction, which directs fluid from the additional solution bag 38e through the inline heater 56 and back to the first solution bag 38b as shown in FIG. 2B. Inline heater 56 is in one embodiment bidirectional, such that PD fluid flowing in either direction from inlet 56i to outlet 560, or from outlet 560 to inlet 56i may be heated to a desired temperature under the control of control unit 100. The control unit 100 continues the operation until the sensed temperature of the PD fluid in the solution bag 38b is above the pre-determined threshold. FIG. 3 shows one method which may be implemented in control unit 100 for preheating the PD fluid prior to fill. At oval 110, the method for preheating the PD fluid begins. At block 112, the control unit 100 may receive a sensed temperature of the PD fluid from the temperature sensor. At diamond 114, the control unit 100 determines whether the temperature of the PD fluid in the solution bag 38b as indicated by the temperature sensor is above or below the pre-determined threshold. When the temperature of the PD fluid is above the pre-determined threshold, the method may end at oval 118 indicating to the control unit 100 that the PD system 10 is ready for patient fill.

At diamond 114, when the temperature of the PD fluid has fallen below the pre-determined threshold, the control unit 100 at block 116 automatically initiates the push pull operation. In some embodiments, initiating the push pull operation includes the control unit 100 opening the third recirculation valve 54r3 and causing the pump 70 to direct fluid from the solution bag 38b, through the inline heater 56, into the additional solution bag 38e, and back as illustrated in FIGS. 2A and 2B. In some embodiments, the control unit 100 will continue the push pull operation until the control unit 100 determines at diamond 114 that the temperature is above the pre-determined threshold. In some embodiments, the control unit 100 will continue the push pull operation until the control unit 100 determines at diamond 114 that the temperature is above a second pre-determined cutoff threshold that may be different than the first pre-determined threshold for initiating the push pull operation. In some embodiments, when the control unit 100 determines the temperature of the PD fluid is above the pre-determined threshold, the control unit 100 will cause the pump 70 to return all of the PD fluid from the additional solution bag 38e to the solution bag 38b prior to fill. Once the control unit 100 determines the temperature is above the threshold, the method will end at oval 118 indicating that the PD system is ready for patient fill.

In some embodiments, the method of FIG. 3 may occur during priming and/or during the dwell period. For example, during priming of the PD system 10, the control unit 100 causes the pump 70 to pump an amount of fluid into the PD system 10 to remove any air and contaminants in the PD system 10 before treatment. The control unit 100 may run the method during the priming phase before treatment.

Additionally or alternatively, the control unit 100 may implement the method during the dwell period. In some embodiments, the control unit 100 implements the method before a last fill, or any other fill cycle of the PD treatment. The control unit 100 may close a patient line valve (e.g. valve 52f or an additional valve) during dwell. At a time before the dwell period ends, such as between 10 minutes and 20 minutes, for example, about 10 minutes, 15 minutes, or 20 minutes before the dwell period ends, the control unit 100 may receive a sensed temperature from the temperature sensor. At diamond 114, the control unit determines whether the sensed temperature is above or below the pre-determined threshold. The control unit 100 may automatically initiate the push pull operation at block 116 when the sensed temperature of the PD fluid in the solution bag 38b is below the pre-determined threshold. This ensures that the initial temperature of the PD fluid will be high enough to permit heating to body temperature using the inline heater 56. Doing so during dwell may prevent down time of treatment for heating of the fluid.

It can be appreciated that during the push pull operation, the PD fluid does not need to be heated to body temperature. However, it is advantageous for the PD fluid in the solution bag to be heated to a lowest initial temperature so that the inline heater 56 can heat the PD fluid to body temperature during fill at the desired fill rate and with a desired inline heater power requirement. Further, since the PD fluid is being preheated and not delivered to the patient, the flow rate of the PD fluid during preheating is less critical.

The above recited device and methods allow the PD fluid to be heated to body temperature with a lower ambient temperature and without increasing the power requirements of the inline heater 56. In some embodiments, the inline heater 56 has a power of less than 650 W, for example between 350 W and 450 W, 450 W and 550 W, or between 550 W and 650 W. Further, the device and methods disclosed herein do not require a decreased fill flow rate to allow the inline heater 56 to heat the PD fluid to body temperature. In some instances, the flow rate of fill may be increased and the therapy time reduced without having to increase the inline heater 56 power. In some embodiments, the flow rate during fill is above 300 ml/min, such as between 300 ml/min and 375 ml/min, between 375 ml/min and 450 ml/min, between 450 ml/min and 525 ml/min, between 525 ml/min and 600 ml/min, and above 600 ml/min.

The invention is claimed as follows:

1. A peritoneal dialysis (“PD”) system comprising:
a housing;
a PD fluid pump housed by the housing;
a source of fresh PD fluid;
an inline heater positioned and arranged to heat the fresh PD fluid;
a fluid line in fluid communication with the source of fresh PD solution, the inline heater, and the PD fluid pump;
a temperature sensor configured to sense a temperature of the source of the fresh PD fluid; and
a control unit configured to control the PD fluid pump, the control unit further configured to:
receive a sensed temperature of the fresh PD fluid from the temperature sensor during a dwell phase or a priming phase of a peritoneal dialysis treatment,
determine whether the sensed temperature is below a pre-determined threshold, and
cause the PD fluid pump to pump the fresh PD fluid through the fluid line and through the inline heater when the sensed temperature is below the pre-determined threshold.

2. The PD system of claim 1, wherein the source of fresh PD fluid comprises a first solution container, and wherein the PD system further comprises a second solution container, and wherein the control unit is configured to cause the PD fluid pump to pump the fresh PD fluid from the first solution container, through the fluid line, through the inline heater, and to the second solution container when the sensed temperature is below the pre-determined threshold.

3. The PD system of claim 2, wherein the control unit is further configured to cause the PD fluid pump to reverse a flow direction of the PD fluid pump and pump the fresh PD fluid from the second solution container to the first solution container and back until the sensed temperature is above the pre-determined threshold.

4. The PD system of claim 1, wherein the temperature sensor is located at the source of the fresh PD fluid.

5. The PD system of claim 1, wherein the temperature sensor is located along the fluid line between the source of fresh PD fluid and the inline heater.

6. The PD system of claim 1, wherein the control unit is further configured to cause the PD fluid pump to begin a patient fill when the sensed temperature is above the pre-determined threshold.

7. The PD system of claim 6, wherein the control unit is further configured to cause the PD fluid pump to begin the patient fill at a flow rate above 300 ml/min.

8. The PD system of claim 1, wherein the inline heater has a power of less than 650 W.

9. The PD system of claim 1, wherein the pre-determined threshold is between 15° C. and 30° C.

10. A peritoneal dialysis (“PD”) method for preheating a PD fluid, the method comprising:
sensing a temperature, via a temperature sensor controlled by a control unit of a PD machine, of fresh PD fluid in a container;
determining, via the control unit, whether the sensed temperature is below a pre-determined threshold;
causing a PD fluid pump of the PD machine, via the control unit, to pump the fresh PD fluid from the container through an inline heater and back when the sensed temperature is below the pre-determined threshold; and
continuing to cause the PD fluid pump, via the control unit, to pump the fresh PD fluid from the container through the inline heater and back until the sensed temperature is above the pre-determined threshold.

11. The PD method of claim 10, further comprising causing a PD pump, via the control unit, to begin a patient fill when the sensed temperature is above the pre-determined threshold.

12. The PD method of claim 11, wherein the patient fill occurs at a flow rate of above 300 ml/min.

13. The PD method of claim 10, wherein the sensing the temperature occurs during a priming phase of a PD treatment as determined by the control unit.

14. The PD method of claim 10, wherein the sensing the temperature occurs during a dwell phase of a PD treatment as determined by the control unit.

15. The PD method of claim 14, further comprising closing a patient line valve, via the control unit, prior to causing the PD fluid pump to pump the fresh PD fluid from the container.

16. The PD method of claim 14, wherein the sensing the temperature occurs between 10 minutes and 20 minutes before an end of the dwell phase.

17. The PD method of claim 10, wherein the pre-determined threshold is between 15° C. and 30° C.

18. The PD method of claim 10, wherein the inline heater has a power of less than 650 W.

19. A peritoneal dialysis (“PD”) system comprising:
a housing;
a PD fluid pump housed by the housing;
a first container comprising a fresh PD fluid;
a second container;
an inline heater positioned and arranged to heat the fresh PD fluid;
a fluid line in fluid communication with the first container, the inline heater, the PD fluid pump, and the second container;
a temperature sensor configured to sense a temperature of the fresh PD fluid; and
a control unit configured to control the PD fluid pump, the control unit further configured to:
receive a sensed temperature of the fresh PD fluid from the temperature sensor during a dwell phase or a priming phase of peritoneal dialysis treatment,
determine whether the sensed temperature is below a pre-determined threshold, and
cause the pump to pump the fresh PD fluid from the first container, through the fluid line, and through the inline heater when the sensed temperature is below the pre-determined threshold.

20. The PD system of claim 19, wherein the control unit is further configured to cause the PD fluid pump to reverse a flow direction of the PD fluid pump and pump the fresh PD fluid from the inline heater to the first container and back until the sensed temperature is above the pre-determined threshold.
